(19) United States
(12) Patent Application Publication
(10) Pub. No.: US 2023/0248991 A1
(43) Pub. Date: Aug. 10, 2023

(54) LIGHT IRRADIATION PROBE AND METHOD OF MANUFACTURING LIGHT IRRADIATION PROBE

(71) Applicant: OLYMPUS CORPORATION, Tokyo (JP)

(72) Inventor: Teppei TSURUTA, Tokyo (JP)

(73) Assignee: OLYMPUS CORPORATION, Tokyo (JP)

(21) Appl. No.: 18/104,843

(22) Filed: Feb. 2, 2023

Related U.S. Application Data
(60) Provisional application No. 63/306,589, filed on Feb. 4, 2022.

(51) Int. Cl. A61N 5/06

(52) U.S. Cl. CPC A61N 5/0601; A61N 5/062; A61N 2005/063; A61N 2005/0666

(57) ABSTRACT

A light irradiation probe comprising:
a catheter having an elongated shape, the catheter being configured to emit light from an emission region arranged on a side surface on a distal end side of the catheter; and wherein the catheter includes an outer sheath having a tubular shape, the outer sheath comprising an outer surface of the catheter; an optical fiber extending in a longitudinal axis direction of the catheter, the optical fiber including a core forming an optical waveguide; and a filling material disposed at a longitudinal position corresponding to the emission region of the outer sheath such that the light emitted from the emission region passes through the filling material, the filling material having a refractive index higher than a refractive index of the core.

### CROSS-REFERENCE TO RELATED APPLICATION

This application is based on and claims priority under 35 U.S.C. § 119 to U.S. Provisional Application No. 63/306,589, filed Feb. 4, 2022, the entire contents of which are incorporated herein by reference.

### BACKGROUND

### 1. Technical Field

The present disclosure relates to a light irradiation probe and a method of manufacturing a light irradiation probe.

### 2. Related Art

In recent years, a study of photoimmunotherapy (PIT) to treat cancer by specifically binding an antibody drug to protein of a cancer cell, and by activating the antibody drug by irradiation of near-infrared light, which is treatment light, to destroy the cancer cell has been developed. The antibody drug to which near-infrared light is irradiated expands a cancer cell, to induce cell death of the cancer cell. At this time, the antibody drug is excited to emit fluorescence. The intensity of this fluorescence is used as indicator for treatment effect.

As a member to irradiate light to a subject, a light irradiation probe in which an optical fiber is inserted thereinside, that guides light from a light source to a distal end portion, and that emits light from the distal end portion has been known (for example, refer to International Publication Pamphlet No. 2020/071023). The light irradiation probe described in International Publication Pamphlet No. 2020/071023 includes a long shaft, a balloon that is arranged at one end portion of the shaft, and a light guiding portion that is movable in a direction of length of the shaft. The light guiding portion includes an optical fiber and a tubular member that covers the optical fiber and that has light transparency. The optical fiber includes a core and a cladding that covers the core, and a light emitting portion in which the cladding is removed is formed in a portion of a distal end of the core. The optical fiber is housed inside the shaft, and processing to diffuse light is applied to a surface of the core of the light emitting portion.

### SUMMARY

Accordingly, a light irradiation probe can include: a catheter having an elongated shape, the catheter being configured to emit light from an emission region arranged on a side surface on a distal end side of the catheter. The catheter includes an outer sheath having a tubular shape, the outer sheath comprising a part of an outer surface of the catheter; an optical fiber extending in a longitudinal direction of length of the catheter, the optical fiber including a core forming an optical waveguide; and a filling material disposed at a longitudinal position corresponding to the emission region of the outer sheath such that the light emitted from the emission region passes through the filling material, the filling material having a refractive index higher than a refractive index of the core.

Also provided is a method of manufacturing a light irradiation probe comprising a catheter having an elongated shape, the catheter being configured to emit light from an emission region arranged on a side surface on a distal end side of the catheter. The method comprising: inserting an optical fiber, a part of which is covered with a cladding, and that has a core with a distal end exposed, into a tubular outer sheath such that the exposed code corresponds to a longitudinal position of the emission region; and filling a filling material made from a material having a refractive index higher than a refractive index of the core in the outer sheath at the longitudinal position corresponding to the emission region such that light emitted from the emission region passes through the filling material.

The above and other features, advantages and technical and industrial significance of this disclosure will be better understood by the following detailed description of presently preferred embodiments of the disclosure, when considered in connection with the accompanying drawings.

### BRIEF DESCRIPTION OF THE DRAWINGS

FIG. 1 is a diagram illustrating a configuration of a light irradiation probe according to a first embodiment;

FIGS. 2(a) and 2(b) are diagrams illustrating one example of a flow of treatment using the light irradiation probe according to the first embodiment;

FIG. 3 is a flowchart illustrating a method of manufacturing the light irradiation probe according to the first embodiment;

FIG. 4 is a diagram illustrating a configuration of a light irradiation probe according to a second embodiment;

FIG. 5 is a flowchart illustrating a method of manufacturing the light irradiation probe according to the second embodiment;

FIG. 6 is a diagram illustrating a configuration of a distal end of an optical fiber of a light irradiation probe according to a third embodiment;

FIG. 7 is a diagram illustrating a schematic configuration of an endoscope system;

FIG. 8 is a diagram explaining a configuration of a distal end of the endoscope;

FIGS. 9(a) and 9(b) are diagrams of an example of application of the light irradiation probe to a needle tube; and

FIGS. 10(a) and 10(b) are diagrams (of an example of application of the light irradiation probe to a needle tube.

### DETAILED DESCRIPTION

Hereinafter, forms to implement the disclosure (hereinafter, “embodiments”) will be explained. In embodiments, a light irradiation probe that is used for photoimmunotherapy (PIT) will be explained as an example of a light irradiation probe according to the disclosure. Moreover, the embodiments are not intended to limit the disclosure. Furthermore, the light irradiation probe will be explained assigning like reference signs to like parts throughout the drawings.

### First Embodiment

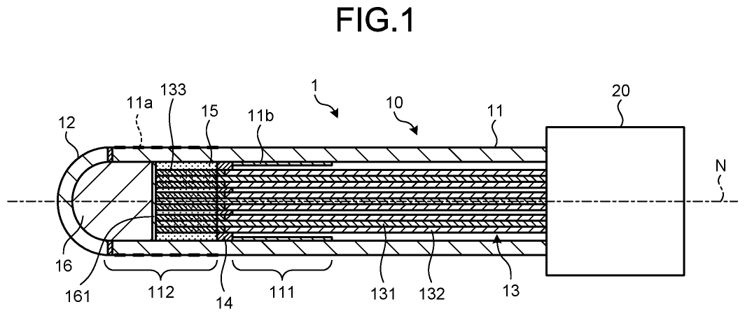

FIG. 1 is a diagram illustrating a configuration of a light irradiation probe according to a first embodiment. A light irradiation probe 1 includes a catheter portion (alternatively referred to as a catheter) 10 and a light source 20.

The catheter portion 10 includes an outer sheath 11, a distal-end sealing portion (alternatively referred to as a distal end seal) 12, multiple optical fibers 13, a partition 14, a filling fluid 15, and a cap portion (alternatively referred to as a cap) 16. An axis extending in a direction of length of the catheter portion 10 is referred to as longitudinal axis N, and a side closer to the light source 20 is referred to as proximal end side and a side opposite to the light source 20 is referred to as distal end side. Moreover, in the catheter portion 10, a bending region 111 configured to be bent relative to the longitudinal axis N and an emission region 112 in which light guided by the optical fiber 13 is emitted outward are provided. The bend region 111 is a portion that is configured to be bent, for example, when it extends out from an opening of a treatment tool channel when the light irradiation probe 1 is inserted into the treatment channel of an endoscope. The bend region 111 is arranged on the proximal end side relative to the partition 14 in this example, but it may be arranged on the distal end side.

One end of the outer sheath 11 is connected to the light source 20, and the other end is connected to the distal-end sealing portion 12. The outer sheath 11 has a tubular shape, and houses the optical fiber 13, the partition 14, the filling fluid 15, and the cap portion 16 thereinside. In this outer sheath 11, on an inner peripheral side of the bend region 111, a metal coating 11b is formed. This metal coating 11b is a coating having a thickness that enables flexible bending. Moreover, in the emission region 112 of the outer sheath 11, a rough portion 11a is formed by applying a surface treatment to form a roughness surface. In the rough portion 11a, light to be emitted outward is diffused by the roughness in a light advancing direction. The outer sheath 11 is formed by using, for example, a light permeable material. The outer sheath 11 may include solid particles and/or air bubbles at least in a position corresponding to the emission region 112. For the solid particles, any particles that can diffuse incident light can be adopted. Moreover, in the emission region 112 of the outer sheath 11, a diffuser plate made from resin, glass, or simple or combined substance of crystalline body, or a concave lens may be arranged. As long as the emission region 112 has light permeability, portions other than the emission region 112 may be impervious. Furthermore, in the outer sheath 11, a region corresponding to the bend region 111 may be formed with a material having high flexibility compared to other portions.

The distal-end sealing portion (alternatively referred to as a distal-end seal or distal-end sealing material) 12 and the cap portion (alternatively referred to as a cap) 16 are arranged in the outer sheath 11 on a side opposite to a side of the light source 20 to liquid-tightly seal one end of the outer sheath 11. The cap portion 16 is arranged inside the distal end portion of the outer sheath 11 in a liquid tight manner. Moreover, the distal-end sealing portion 12 is connected to the distal end of the outer sheath 11, and is joined at a connection. In the first embodiment, an example in which a distal end surface of the distal end sealing portion 12 is a curved surface is explained, but it may have other shapes, such as planar shape.

The optical fiber 13 is an elongated light guiding member that guides light of the light source 20 to the emission region 112. The optical fiber 13 includes a core 131 comprising a central portion, a cladding 132 and a cover 133 that cover respectively different portions of a surrounding area of the core 131. The refractive index of the core 131 is higher than the refractive index of the cladding 132. Moreover, the refractive index of the cover 133 is higher than the refractive index of the core 131. For example, in the optical fiber 13 covered with the cladding 132, light is propagated in a state in which the light is confined in the core 131 by making the cladding 132 entirely reflect light. On the other hand, in the optical fiber 13 covered with the cover 133, light from the core 131 is not entirely reflected by the cover, light from the core 131 is emitted outside of the optical fiber 13.

Multiple pieces of the optical fibers 13 may be covered with a tubular-shaped or spiral-shaped cover material. Also when only one piece of the optical fiber 13 is provided, it may be configured to be covered with the cover material described above. Moreover, the light irradiation probe 1 can have a configuration in which multiple pieces of the very thin optical fibers 13 are bundled in terms of breaking resistance and damage suppression.

The partition 14 is arranged between the bend region 111 and the emission region 112 in a direction of the longitudinal axis N, and divides an interior of the outer sheath 11. The optical fiber 13 comprises the core 131 and the cladding 132 on a proximal end side relative to the partition 14, and comprises the core 131 and the cover 133 on a distal end side relative to the partition 14. In the first embodiment, an example in which the partition 14 is made from ultraviolet-curing resin is explained. The ultraviolet-curing resin includes curing resin of acrylic resin and epoxy resin. Other than ultraviolet-curing resin, solvent-curing type resin, moisture-curing type resin, and heat-curing type resin can be used. The solvent-curing type resin includes curing resins of vinyl acetate, and nitrile rubber. The moisture-curing type resin includes curing resins of cyanoacrylate, and silicon rubber. The heat curable resin includes curable resin of epoxy resin and acrylic resin.

The filling fluid 15 is filled in a space formed with the outer sheath 11, the partition 14 and the cap portion 16. The filling fluid 15 has light permeability, and fluid having higher refractive index than the core 131 and the cover 133 is used. Furthermore, in the outer sheath 11, a refractive index in at least the emission region 112 is higher than refractive index of the filling fluid. The filling fluid 15 may include solid particles and/or air bubbles. As the solid particles, any particle that diffuses incident light can be adopted. Moreover, in terms of diffusing light efficiently, the density of the solid particles or air bubbles can become higher as it approaches a distal end of a catheter portion. Generally, light leakage (light intensity) from the core 131 is larger on a light source side (proximal end side) than that on the opposite side to the light source (distal end side). Therefore, by increasing the density of solid particles or the like on the distal end side, a difference in uniformity between the distal end side and the proximal end side can be decreased.

The cap portion 16 is pressure-bonded to an inner peripheral surface of the outer sheath 11, and is intimately connected to the outer sheath 11 in a liquid tight manner. The cap portion 16 is formed by using stretchable material (for example, rubber), and is press-fitted into the outer sheath 11 by forming it into the size larger than that of an opening of the outer sheath 11 in a natural state when a load other than gravity is not applied, or a solid material larger than the opening of the outer sheath 11 is press-fitted into the outer sheath 11. When the cap portion 16 is manufactured with, for example, rubber, the filling fluid 15 can be refilled by puncturing an injection syringe.

On a surface that is a proximal end surface of the cap portion 16 facing the optical fiber 13, a mirror 161 is arranged. In the first embodiment, a distal end (the core 131 and the cover 133 in this case) of each of the optical fibers 13 abuts on the mirror 161. The mirror 161 may have a configuration in which a surface facing the optical fiber 13 is curved to have a convex shape, or multiple convex shapes, to diffuse incident light.

The light source 20 is a treatment-light emitting unit that is used for, for example, PIT, and that includes a light source to emit light for treatment (hereinafter, treatment light). The light source 20 emits treatment light, for example, when a switch or the like is pressed. The light source 20 may be configured to emit light under control of an external control device. The light source equipped in the light source 20 is implemented by using a semiconductor laser, an LED, or the like. For example, in the case of PIT, the treatment light is light having a wavelength band of 680 nm or longer, and is light having a center wavelength of, for example, 690 nm.

An illumination optical system included in the light source 20 may be configured to be capable of changing an irradiation range of the treatment light. For example, by applying an optical system that can change a focal length, a digital micromirror device (DMD), or the like, intensity distribution of light to be irradiated to a subject, or a shape of irradiation range can be changed.

Moreover, the light source 20 may be fixed to the catheter portion 10, or may be detachable with respect to the catheter portion 10. When the light source 20 is detachable with respect to the catheter portion 10, it is possible to withdraw the endoscope, leaving only the catheter portion 10 inside the body of a patient. Furthermore, after withdrawal of the endoscope, the catheter portion 10 and the light source 20 can be connected, to continue irradiation of the treatment light. Moreover, by making it detachable, the catheter portion 10 can be disposable. In this case, in at least one of the catheter portion 10 and the light source 20, by arranging a permanent magnet at an end portion on a connecting side of the catheter portion 10 and the light source 20 to establish a mechanism to draw the other by the magnetic force, it is possible to ensure connection between the catheter portion 10 and the light source 20.

In the light irradiation probe 1, light emitted from the light source 20 is taken into the core 131 of the optical fiber 13, and propagates to the distal end side. The light advancing to the distal end side passing through the partition 14 on the direction of the longitudinal axis N is emitted outward from the emission region 112 through the cover 133 and the filling fluid 15 from the core 131. At this time, because light travels through a path in which the refractive index gradually increases from the core 131 to the outer sheath 11 (the emission region 112) through the cover 133 and the filling fluid 15, the light is emitted from the outer sheath 11 at a high extraction efficiency without causing an entire reflection or the like. In this case, light is diffused at the rough portion 11a of the outer sheath 11, and is emitted outward in a wide range.

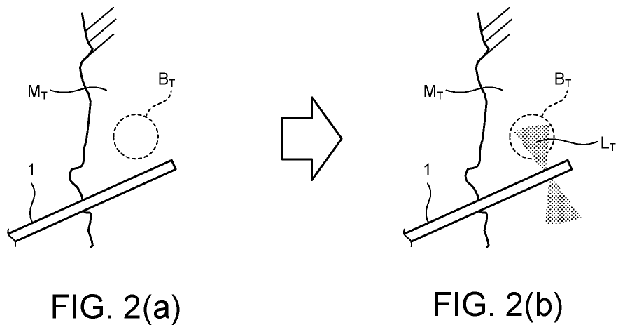

Subsequently, a flow of treatment using the light irradiation probe will be explained referring to FIGS. 2(a) and 2(b). FIGS. 2(a) and 2(b) are a diagrams illustrating one example of a flow of treatment using the light irradiation probe according to the first embodiment. FIGS. 2(a) and 2(b) are diagrams illustrating one example of implementation of PIT, and illustrates an example in which treatment is performed by inserting the light irradiation probe 1 into the body of a subject.

First, an operator places the light irradiation probe 1 at a position in which a tumor to be treated of a subject MT is present (refer to FIG. 2(a)). In this example, treatment of a tumor BT present under the skin of the subject MT is performed. The light irradiation probe 1 is positioned at a position at which the tumor is present, for example, through a stent, a needle tube, or a treatment tool channel of an endoscope. At this time, administration of an antibody drug is performed with respect to the tumor BT, which is an area to be treated. Administration of the antibody drug may be performed by using an endoscope or the like, or by using other devices, or may have the patient swallow a drug.

The operator directs the emission region 112 of the light irradiation probe 1 toward the tumor BT, and irradiates treatment light LT to the tumor BT (refer to FIG. 2(b)). By the irradiation of the treatment light, the antibody drug bound with the tumor BT reacts, and treatment for the tumor BT is performed. The operator checks a treatment effect in the tumor BT by acquiring, for example, a fluorescence image after treatment.

The operator repeats additional irradiation of treatment light and checks treatment effect as necessary.

By performing the processing described above, treatment of a tumor is performed by the light irradiation probe 1.

Even if light cannot be entirely reflected by the cladding 132 and leaks outside as the catheter portion 10 is bent in the bend region 111 at the time of use, the leaked light is reflected by the metal coating 11b, and returns to the core 131. Moreover, light emitted from the distal end of the core 131 is reflected by the mirror 161, and returns to the core 131.

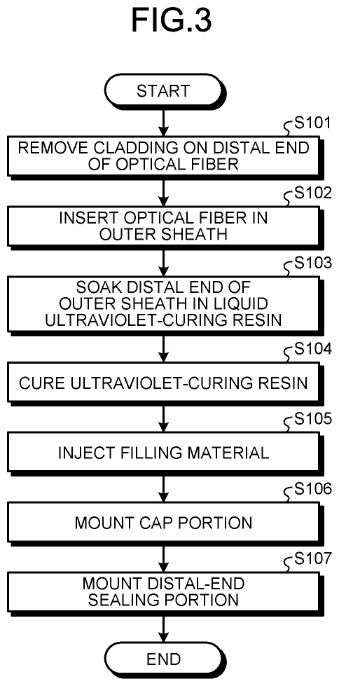

Subsequently, a method of manufacturing the light irradiation probe 1 will be explained, referring to FIG. 3. FIG. 3 is a flowchart illustrating the method of manufacturing the light irradiation probe according to the first embodiment.

First, the cladding 132 at one end of the optical fiber 13 is removed (step S101). In the first embodiment, the cover 133 is arranged in a portion from which the cladding 132 is removed.

After removing the cladding 132, the optical fiber 13 is inserted in the outer sheath 11 (step S102).

A distal end (on a side on which the cover 133 is arranged) of the outer sheath 11 is soaked in liquid ultraviolet-curing resin (step S103). After soaking, the ultraviolet-curing resin at a partition forming position (between the cladding 132 and the cover 133 in this example) of the outer sheath 11 is cured, to form the partition 14 (step S104).

After formation of the partition 14, the filling fluid 15 is injected into the outer sheath 11 (step S105). After injection of the filling fluid 15, the cap portion 16 is mounted on the distal end of the outer sheath 11 (step S106). Furthermore, the distal-end sealing portion 12 is mounted on the distal end of the distal end of the outer sheath 11, and the distal-end sealing portion 12 is joined with the outer sheath 11 (step S107). By the procedure described above, the light irradiation probe 1 is manufactured. In the course of the processing described above, or after the processing, processing of the surface treatment of the outer sheath 11 is performed to form the rough portion 11a, and the metal coating 11b is formed on the outer sheath 11 before the optical fiber 13 is inserted.

In the first embodiment explained above, in the light irradiation probe 1, the core 131 is covered with the cladding 132 in a traveling path of light before emission of light, and leakage of light from the core 131 is thereby prevented, and after passing the partition 14, because the refractive index of the respective members gradually increases in a traveling path of light from the core 131 to the outer sheath 11, light can be efficiently extracted out from the outer sheath 11. According to the first embodiment, light from an optical fiber can be extracted highly efficiently, to be emitted.

Moreover, in the first embodiment, because the emission region 112 is arranged on a side surface portion of the catheter portion 10 so that a surrounding area perpendicular to a direction of length of the catheter portion 10 is to be subject to irradiation, the irradiation range of light can be wider than a case in which light is emitted from a distal end of an optical fiber.

### Second Embodiment

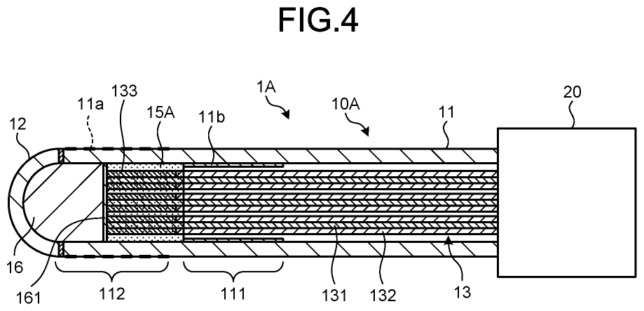

Next, a second embodiment will be explained. FIG. 4 is a diagram illustrating a configuration of a light irradiation probe according to the second embodiment. A light irradiation probe 1A according to the second embodiment includes a filling material 15A in place of the partition 14 and the filling fluid 15 of the light irradiation probe 1 according to the first embodiment. Because components other than the filling material 15A are the same as the light irradiation probe 1, explanation thereof will be omitted.

The light irradiation probe 1A includes a catheter portion 10A and the light source 20.

The catheter portion 10A includes the outer sheath 11, the distal-end sealing portion 12, the optical fiber 13, the filling material 15A, and the cap portion 16. In the catheter portion 10A, similarly to the first embodiment, the bend region 111 that is configured to be bent and the emission region 112 in which light guided by the optical fiber 13 is emitted outward are provided.

The filling material 15A is arranged on the distal end side of the outer sheath 11. The filling material 15A has light permeability, and a material having a refractive index higher than the core 131 and the cover 133, and a refractive index lower than the outer sheath 11 (the emission region 112) is used. The filling material 15A extends in the direction of the longitudinal axis N from the proximal end side of the cap portion 16 to the distal end of the bend region 111. The optical fiber 13 comprises the core 131 and the cladding 132 on a proximal end side relative to the emission region 112, and comprises the core 131 and the cover 133 on a distal end side relative to proximal end of the emission region 112. The filling material 15A has functions of the partition 14 and the filling fluid 15 in the first embodiment. The filling material 15A is made from a material, a refractive index of its inside of which is higher than the cover 133 and lower than the outer sheath 11. In the second embodiment, an example in which the filling material 15A is made from ultraviolet-curing resin is explained. The ultraviolet-curing resin includes curing resin of acrylic resin and epoxy resin. Other than ultraviolet-curing resin, heat-curing type resin, curing-agent-mixed type resin, and heat-melting type resin can be used. The heat curing type resin includes curable resin of epoxy resin and acrylic resin. The curing-agent mixed type resin includes curing resins of epoxy resin, silicon rubber, and acrylic resin. The heat-melting type resin includes curing resin of styrene-butadiene rubber. Moreover, the filling material 15A may include solid particles and/or air bubbles. For the solid particles, any particles that can diffuse incident light can be adopted.

In the light irradiation probe 1A, light emitted from the light source 20 is taken into the core 131 of the optical fiber 13, and propagates to the distal end side. The light advancing to the distal end side passing through the proximal end of the emission region 112 in the longitudinal direction N is emitted outward from the emission region 112 through the cover 133 and the filling material 15A from the core 131. At this time, because light travels through a path in which the refractive index gradually increases in a portion from the core 131 to the outer sheath 11 (the emission region 112) through the cover 133 and the filling material 15A, the light is emitted from the outer sheath 11 at a high extraction efficiency without causing an entire reflection or the like. In this case, light is diffused at the rough portion 11a of the outer sheath 11, and is emitted outward in a wide range.

Even if light cannot be entirely reflected by the cladding 132 and leaks outside as the catheter portion 10 is bent in the bend region 111 at the time of use, the leaked light is reflected by the metal coating 11b, and returns to the core 131. Moreover, light emitted from the distal end of the core 131 is reflected by the mirror 161, and returns to the core 131.

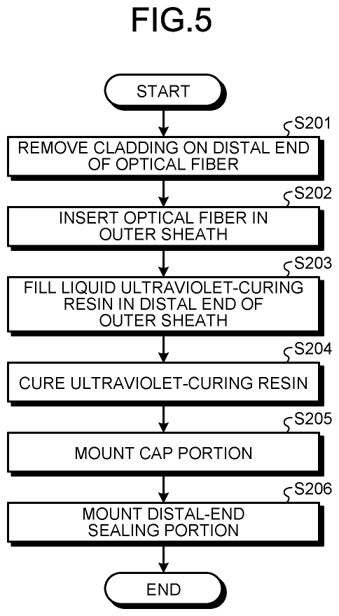

Subsequently, a method of manufacturing the light irradiation probe 1A will be explained, referring to FIG. 5.

FIG. 5 is a flowchart illustrating the method of manufacturing the light irradiation probe according to the second embodiment.

First, the cladding 132 at one end of the optical fiber 13 is removed similarly to steps S101 and S102 described above, and the optical fiber 13 is inserted in the outer sheath 11 (steps S201, S202).

Thereafter, liquid ultraviolet-curing resin is filled at the distal end of the outer sheath 11 (on the side on which the cover 133 is arranged) (step S203). After filling, the ultraviolet-curing resin on the distal end side from the partition forming position (between the cladding 132 and the cover 133 in this example) of the outer sheath 11 is cured, to form the filling material 15A (step S204: curing step).

After formation of the filling material 15A, the cap portion 16 is mounted on the distal end of the outer sheath 11 (step S205). Furthermore, the distal-end sealing portion 12 is mounted on the distal end of the outer sheath 11, and the distal-end sealing portion 12 is joined with the outer sheath 11 (step S206). By the procedure described above, the light irradiation probe 1A is manufactured.

In the second embodiment explained above, in the light irradiation probe 1, the core 13 is covered with the cladding 132 in a traveling path of light before emission of light, and leakage of light from the core 13 is thereby prevented, and after passing the position of the proximal end of the emission region 112, because the refractive index of the respective members gradually increases in a traveling path of light from the core 131 to the outer sheath 11, light can be efficiently extracted out from the outer sheath 11. According to the second embodiment, light from an optical fiber can be extracted highly efficiently, to be emitted.

Moreover, according to the second embodiment, because the partition 14 and the filling fluid 15 according to the first embodiment are replaced with the unified filling material 15A, and it is formed by curing this filling material 15A, leakage of liquid (the filling fluid 15) does not occur, and handling at the time of use and at the time of manufacturing becomes easy.

### Third Embodiment

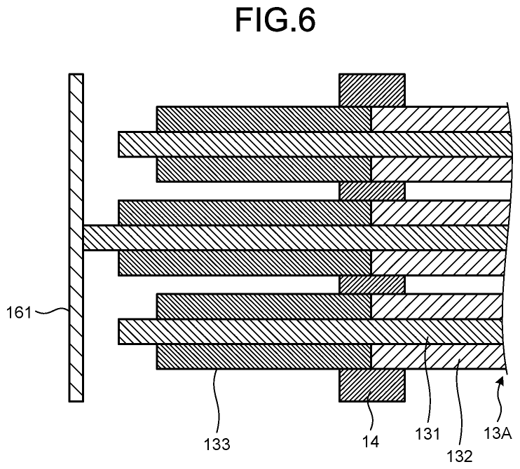

Next, a third embodiment will be explained. FIG. 6 is a diagram illustrating a configuration of a distal end of an optical fiber of a light irradiation probe according to a third embodiment. The light irradiation probe according to the third embodiment differs in the arrangement of an optical fiber 13A in the light irradiation probe 1 according to the first embodiment. Because configuration other than the arrangement of the optical fiber 13 is the same as the light irradiation probe 1, explanation thereof will be omitted.

In the respective optical fibers 13A according to the third embodiment, a part of the core 131 is covered with the cover 133, and a distal end thereof is exposed. Furthermore, the respective optical fibers 13A have different distal end positions on the distal end side. Specifically, in an example illustrated in FIG. 6, while a distal end of some of the optical fibers 13A abuts on the mirror 161, distal ends of the other optical fibers 13A are apart from the mirror 16. For example, in a group of the multiple optical fibers 13A, positions of distal ends of the respective optical fibers 13A are offset outward from the center optical fiber 13A that abuts on the mirror 161.

In the third embodiment explained above, similarly to the first embodiment, light from an optical fiber can be extracted highly efficiently, to be emitted.

Moreover, according to the third embodiment, because the distal end positions of the respective optical fibers 13A are offset, light emission positions and the like differ from one another. Consequently, further uniformly distributed emission light can be obtained.

### Other Embodiments

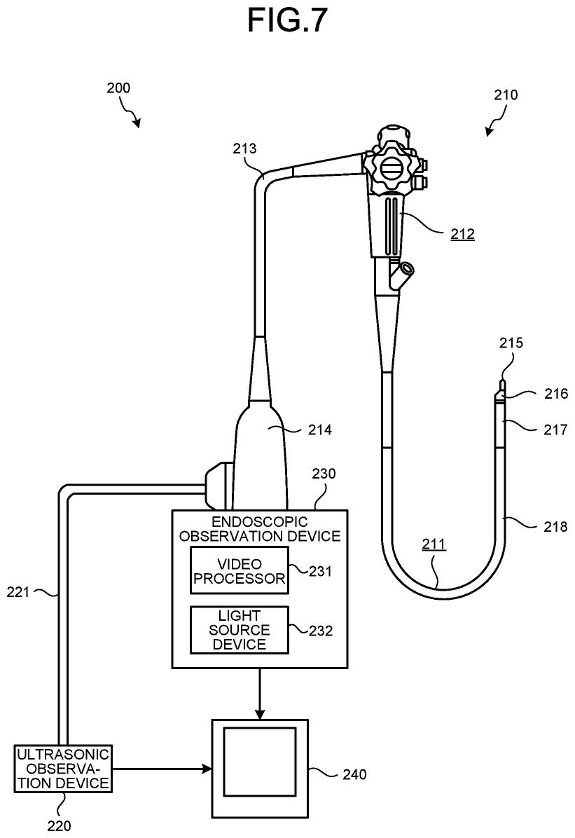

Next, a usage example of the light irradiation probe will be explained, referring to FIG. 7 to FIGS. 10(a) and 10(b).

Usage Example in Endoscope System

FIG. 7 is a diagram illustrating a schematic configuration of an endoscope system. A light irradiation probe is inserted in the body of a subject through an endoscope included in the endoscope system, and irradiates light to a treatment site in the body of the subject. In the following, an example in which the light irradiation probe 1 is used will be explained, but a light irradiation probe of other types may be used also.

An endoscope system 200 illustrated in FIG. 7 is a system that performs ultrasonic diagnosis of the inside of a subject, such as a human, by using an ultrasound endoscope. This endoscope system 200 includes an ultrasound endoscope 210, an ultrasonic observation device 220, an endoscopic observation device 230, and a display device 240.

The ultrasound endoscope 210 corresponds to an endoscope according to the disclosure. This ultrasound endoscope 210 can be partially inserted into the body of a subject, and has a function of outputting an echo signal by transmitting an ultrasound pulse (acoustic pulse) toward a body wall inside the subject and receiving an ultrasound echo reflected on the subject, and a function of outputting an image signal by capturing an image of the inside of the subject.

The ultrasonic observation device 220 is electrically connected to the ultrasound endoscope 210 through an ultrasound cable 221, and outputs a pulse signal to the ultrasound endoscope 210 through the ultrasound cable 221 and receives an echo signal from the ultrasound endoscope 210 as an input. In the ultrasonic observation device 220, the echo signal is subjected to predetermined processing to generate an ultrasound image.

To the endoscopic observation device 230, an endoscope connector 214 described later of the ultrasound endoscope 210 is detachably connected. This endoscopic observation device 230 includes a video processor 231 and a light source device 232.

The video processor 231 receives an image signal from the ultrasound endoscope 210 as an input through the endoscope connector 214. The video processor 231 generates an endoscopic image by subjecting the image signal to predetermined processing.

The light source device 232 supplies illumination light to illuminate the inside of the subject to the ultrasound endoscope 210 through the endoscope connector 214.

The display device 240 comprises a liquid crystal or an organic electroluminescence (EL), and displays an ultrasound image generated by the ultrasonic observation device 220, an endoscopic image generated by the endoscopic observation device 230, and the like.

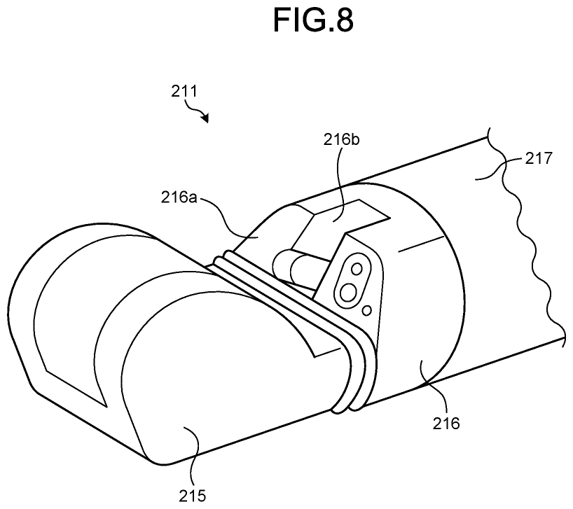

The ultrasound endoscope 210 includes an insertion portion 211, an operating portion 212, a universal cord 213, and the endoscope connector 214. FIG. 8 is a diagram explaining a configuration of a distal end of the endoscope. In the following, in explanation of a configuration of the insertion portion 211, a distal end side of the insertion portion 211 (distal end side in an inserting direction to the body of a subject) is referred to as simply “distal end side”, and a proximal end side of the insertion portion 211 (side departing from the distal end of the insertion portion 211) is referred to as simply “proximal end side”.

The insertion portion 211 is a portion that is inserted in to the body of a subject. This insertion portion 211 includes an ultrasound probe 215 that is arranged on the distal end side, a rigid portion 216 that is connected to the proximal end side of the ultrasound probe 215, a bend portion 217 that is connected to a proximal end side of the rigid portion 216 and is configured to be bent, and a flexible tube 218 that is connected to a proximal end side of the bend portion 217 and is flexible.

The ultrasound probe 215 is a convex ultrasound probe, and has multiple piezoelectric devices that are regularly arranged to form a convex arc. The ultrasound probe 215 converts a pulse signal input from the ultrasonic observation device 220 through the ultrasound cable 221 and a transducer cable into an ultrasonic pulse, to transmit to the inside of the subject. Moreover, the ultrasound probe 215 converts an ultrasonic echo reflected on the inside of the subject into an electrical echo signal, to output to the ultrasonic observation device 220 through the transducer cable and the ultrasound cable 221.

The rigid portion 216 is a rigid portion comprising a resin material, or the like, and has a substantially cylindrical shape that extends linearly. On a distal end side on an outer surface of the rigid portion 216, a flat-shaped inclined surface 216a that makes the rigid portion 216 have a tapered shape toward the distal end is formed. On this inclined surface 216a, an observation window (objective lens) and an illumination lens are arranged.

Moreover, in the rigid portion 216, a treatment tool channel 216b that pierces through from the proximal end to the inclined surface 216a is formed. In addition, in the rigid portion 216, an air/water feeding hole, and the like are formed.

In the endoscope system 200, under observation by the ultrasound endoscope 210, the light irradiation probe 1 is inserted in to the insertion portion 211 from a treatment-tool insertion opening, and the distal end portion of the light irradiation probe 1 (for example, the emission region 112) is placed at a treatment site present thereoutside (inside the subject) from the distal end of the insertion portion 211 through the treatment tool channel 216b. A user operates the light irradiation probe while observing endoscopic images, and irradiates the treatment light to the treatment site.

[Usage Example Using Needle Tube (Part 1)]

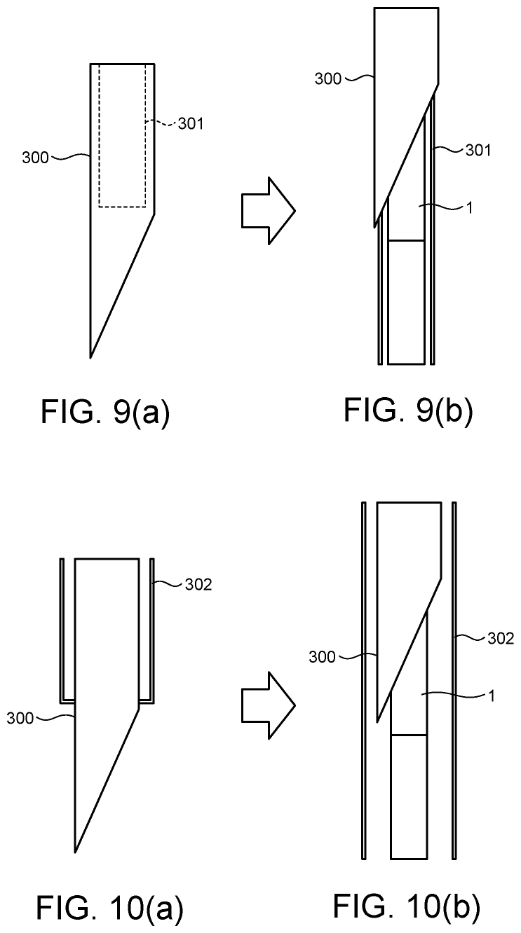

FIGS. 9(a) and 9(b) are diagrams (Part 1) explaining an example of application of the light irradiation probe to a needle tube. In FIGS. 9(a) and 9(b), the light irradiation probe is inserted in a needle tube, and irradiates treatment light to a treatment site through the needle tube. The needle tube may be punctured directly into a subject, or may be introduced into a subject through the treatment tool channel 216b of the ultrasound endoscope 210 described above, a stent, or the like. In the following, an example of using the light irradiation probe 1 will be explained, but other types of light irradiation probe may also be used.

A needle tube 300 is, for example, a biopsy needle, and is a tubular member having a sharp distal end shape. The needle tube 300 includes an inner tube 301 that can move inside the needle tube 300, and a part of which can protrude outward from a distal end of the needle tube 300 (refer to FIG. 9(a)). At least a portion of the inner tube 301 in a position corresponding to the emission region 112 of the light irradiation probe 1 is made from a material that passes light of a wavelength band emitted by the light irradiation probe 1, or a transparent material. The light irradiation probe 1 is arranged to be able to move back and forth with respect to the needle tube 300, and is positioned inside the inner tube 301. The light irradiation probe 1 and the inner tube 301 may be configured to move integrally with respect to the needle tube 300, or may be configured to move independently of each other.

When treatment is performed by using the light irradiation probe 1, first, the needle tube 300 is punctured at a treatment site or its adjacent area in a state in which the inner tube 301 and the light irradiation probe 1 are housed in the needle tube 300 (state in which the inner tube 301 and the light irradiation probe 1 are not protruding from the distal end of the needle tube 300).

Thereafter, by drawing the needle tube 300 and pushing the inner tube 301 forward, the inner tube 301 and the light irradiation probe 1 are made to protrude from the distal end of the needle tube 300 (refer to FIG. 9(b)). By emitting light from the emission region 112 of the light irradiation probe 1 in this state, light irradiation to the treatment site is performed. Moreover, because the inner tube 301 suppresses interference between the light irradiation probe 1 and the needle tube 300, damage of the light irradiation probe 1 caused by the needle tube 300 can be thereby suppressed.

[Usage Example Using Needle Tube (Part 2)]

FIGS. 10(a) and 10(b) are diagrams (Part 2) explaining about an example of application of the light irradiation probe to a needle tube. In FIGS. 10(a) and 10(b), the light irradiation probe is inserted in a needle tube, and irradiates treatment light to a treatment site through the needle tube. The needle tube may be punctured directly into a subject, or may be introduced into a subject through the treatment tool channel 216b of the ultrasound endoscope 210 described above, a stent, or the like. In the following, an example of using the light irradiation probe 1 will be explained, but other types of light irradiation probe may also be used.

In FIGS. 10(a) and 10(b), a needle tube 300 is a tubular member having a sharp distal end shape. The needle tube 300 includes an outer tube 302 that covers an outer portion of the needle tube 300, and that can move in an extending direction of the needle tube 300 (refer to FIG. 10(a)). In FIGS. 10(a) and 10(b), the outer tube 302 is illustrated in cross-section. At least a portion of the outer tube 302 in a position corresponding to the emission region 112 of the light irradiation probe 1 is made from a material that passes light of a wavelength band emitted by the light irradiation probe 1, or a transparent material. The light irradiation probe 1 is arranged to be able to move back and forth inside the needle tube 300.

When treatment is performed by using the light irradiation probe 1, first, the needle tube 300 is punctured at a treatment site or its adjacent area in a state in which the light irradiation probe 1 are housed in the needle tube 300 while positioning the distal end of the outer tube 302 on the proximal end side relative to the distal end of the needle tube 300.

Thereafter, by drawing the needle tube 300 and pushing the outer tube 302 forward, the outer tube 302 and the light irradiation probe 1 are made to protrude from the distal end of the needle tube 300 (refer to FIG. 10(b)). By emitting light from the emission region 112 of the light irradiation probe 1 in this state, light irradiation to the treatment site is performed. Moreover, because the outer tube 302 suppresses bend of the light irradiation probe 1 when the light irradiation probe 1 protrudes out from the needle tube 300, to suppress interference between the light irradiation probe 1 and the needle tube 300, damage of the light irradiation probe 1 caused by the needle tube 300 can be thereby suppressed.

The embodiments to implement the disclosure have so far been explained, but the disclosure is not to be limited to the embodiments described above. For example, although a configuration in which the light irradiation probe 1 includes the light source 20 has been explained in the first embodiment described above, the light irradiation probe may be configured without the light source 20 and configured such that the optical fiber 13 takes in light supplied by an external light source device.

Moreover, although a configuration in which the optical fibers 13, 13A are covered with the cover 133 on the distal end side has been explained in the first to the third embodiments described above, a configuration without the cover 133 may be applied. For example, if it is configured without the cover 133 in the first embodiment, the core 131 directly contacts the filling fluid 15.

Furthermore, a metal electrode portion exposing outside may be arranged at a distal end of a light irradiation probe, and the metal electrode portion and an external high-frequency power source may be electrically connected through an electric wire. In this configuration, the metal electrode portion outputs a high frequency as it is energized.

Moreover, an opening to insert a guide wire may be formed at a distal end of a light irradiation probe.

In the configuration including the metal electrode portion or the guide wire insertion opening, a distal end of a catheter portion may be formed in a tapered shape.

Furthermore, in a light irradiation probe, a lumen having a hole shape extending from a proximal end to a distal end of the light irradiation probe may be formed. Moreover, a balloon may be attached to the emission region 112 of a light irradiation probe, and a supply-and-drainage lumen that has a hole shape extending from a proximal end of the light irradiation probe to a position at which the balloon is attached, and that lets a liquid to inflate the balloon flow may be formed.

Furthermore, a fall prevention function may be provided to a light irradiation probe, for example, by giving a shape memory of bending shape (bend) to a distal end, or by forming a flap mechanism.

Moreover, a light irradiation probe may be provided with a radiopaque marker or an ultrasound strong reflection marker at a distal end.

Furthermore, in the light irradiation probe, the light source 20 may include a primary battery or a secondary battery. In addition, if the buttery to be mounted is compact, even when the light irradiation probe is indwelled in a patient, the patient can move freely.

Moreover, in a light irradiation probe, the light source 20 may include a control circuit having an input unit that accepts an input of ON/OFF switching timing for output of the light source 20, and the like, a display unit that displays the input information, a memory that accepts the input information and various kinds of programs, and a timer that performs time adjustment of the switching timing or the like.

Furthermore, a light irradiation probe may include a temperature sensor arranged at a distal end of a catheter portion, and may perform feedback control on output of a light source based on information from the temperature sensor. At this time, the temperature sensor transmits an electrical signal to a feedback circuit provided on the proximal end side through a signal line. This electrical signal has, for example, an increased intensity (amplitude) according to temperature, and the feedback circuit performs feedback control based on the intensity of the signal. By performing feedback control, treatment can be performed without operating the light irradiation probe regularly by a doctor or the like, and low temperature burns caused by an increase of temperature, or the like can be suppressed.

As described above, a light irradiation probe and a method of manufacturing a light irradiation probe according to the disclosure are useful for extracting light from an optical fiber efficiently to emit it.

According to the disclosure, an effect that light from an optical fiber can be efficiently extracted and emitted is produced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A light irradiation probe comprising:
a catheter having an elongated shape, the catheter being configured to emit light from an emission region arranged on a side surface on a distal end side of the catheter; and
wherein
the catheter includes
an outer sheath having a tubular shape, the outer sheath comprising an outer surface of the catheter;
an optical fiber extending in a longitudinal axis direction of the catheter, the optical fiber including a core forming an optical waveguide; and
a filling material disposed at a longitudinal position corresponding to the emission region of the outer sheath such that the light emitted from the emission region passes through the filling material, the filling material having a refractive index higher than a refractive index of the core.

2. The light irradiation probe according to claim 1, wherein
the outer sheath has a higher refractive index at least at the longitudinal position corresponding to the emission region than the refractive index of the filling material.

3. The light irradiation probe according to claim 1, further comprising a light source connected to a proximal end of the catheter, the light source being configured to supply light to the optical fiber.

4. The light irradiation probe according to claim 1, wherein
the optical fiber includes a cover configured to cover the core in the longitudinal position corresponding to the emission region, the cover having a refractive index higher than the refractive index of the core.

5. The light irradiation probe according to claim 1, wherein
at least one of the outer sheath and the filling material includes one or more of solid particles and air bubbles.

6. The light irradiation probe according to claim 5, wherein
a density of one or more of the solid particles and the air bubbles increases distally.

7. The light irradiation probe according to claim 1, wherein
the catheter further comprises
a distal-end seal arranged at a distal end of the outer sheath, the distal-end seal being configured to seal a distal opening of the outer sheath.

8. The light irradiation probe according to claim 7, further comprising
a mirror arranged between the distal-end seal and a distal end of the optical fiber.

9. The light irradiation probe according to claim 8, wherein
the mirror has a convex shape relative to the distal end of the optical fiber.

10. The light irradiation probe according to claim 8, wherein
the distal end of the optical fiber is configured to abut on the mirror.

11. The light irradiation probe according to claim 1, further comprising
a concave lens arranged at the longitudinal position corresponding to the emission region.

12. The light irradiation probe according to claim 1, wherein
the optical fiber comprises multiple optical fibers, and
longitudinal positions of a distal end surface of the respective multiple optical fibers are offset in an outward radial direction.

13. The light irradiation probe according to claim 1, wherein
the catheter further comprises
a partition arranged inside the outer sheath and on a proximal end side of the emission region, the partition being configured to divide an internal space of the outer sheath.

14. The light irradiation probe according to claim 13, wherein the catheter further comprises
a bending region in which the catheter is configured to be bent is arranged on a proximal end side of the partition, and
in the catheter, a metal coating is formed between the outer sheath and the optical fiber in the bending region.

15. The light irradiation probe according to claim 14, wherein
in the bending region, the catheter is made from a material having higher flexibility than a portion of the catheter proximal to the bending region.

16. The light irradiation probe according to claim 1, wherein
the optical fiber comprises one or more optical fibers, and
the one or more optical fibers are covered with a cover material having any one of a tubular shape and a spiral shape.

17. A method of manufacturing a light irradiation probe comprising a catheter having an elongated shape, the catheter being configured to emit light from an emission region arranged on a side surface on a distal end side of the catheter portion, the method comprising:
inserting an optical fiber, a part of which is covered with a cladding, and that has a core with a distal end exposed, into a tubular outer sheath such that the exposed core corresponds to a longitudinal position of the emission region; and
filling a filling material made from a material having a refractive index higher than a refractive index of the core in the outer sheath at the longitudinal position corresponding to the emission region such that light emitted from the emission region passes through the filling material.

18. The method of manufacturing a light irradiation probe according to claim 17, wherein
the filling material is in a liquid form, the method further comprising
soaking an end portion of the outer sheath before the filling material is filled in liquid curing resin; and
forming a partition by curing the liquid curing resin positioned on a proximal end side relative to the emission region, wherein
the filling includes filling the filling material in the outer sheath in a liquid form on a distal end side relative to the partition.

19. The method of manufacturing a light irradiation probe according to claim 17, wherein
the filling material is made from curing resin, the method further comprising:
soaking the exposed core in liquid curing resin; and
curing the curing resin in the emission region.

20. The method of manufacturing a light irradiation probe according to claim 17, further comprising covering the exposed core with a cover having a refractive index higher than a refractive index of the core.
